(19) United States
(12) Patent Application Publication
(10) Pub. No.: US 2016/0199019 A1
(43) Pub. Date: Jul. 14, 2016

(54) METHOD AND APPARATUS FOR FOCAL SPOT POSITION TRACKING

(71) Applicant: Arineta Ltd., Caesarea (IL)

(72) Inventors: David RUIMI, Ganot Hadar (IL); Lana VOLOKH, Haifa (IL); Evgeniy KUKSIN, Haifa (IL)

(21) Appl. No.: 14/595,564

(22) Filed: Jan. 13, 2015

(51) Int. Cl. A61B 6/00; A61B 6/03

(52) U.S. Cl. CPC A61B 6/547; A61B 6/03; A61B 6/4291

(57) ABSTRACT

A tracking system for tracking focal spot position of an x-ray source includes a detector array including a plurality of detecting elements sensitive to x-ray radiation and a grid overlaid on a detecting surface of the detector array. The grid is formed from an array of vanes and has a density greater than a density of the detector array.

### FIELD AND BACKGROUND OF THE INVENTION

The present invention, in some embodiments thereof, relates to focal spot position tracking of a radiation source and, more particularly, but not exclusively, to focal spot position tracking for radiation scanners, such as computed tomography (CT) scanners and the like.

CT scanners have found wide application in many areas including quality inspection, security and medical imaging. CT scanners typically include a radiation source and a detector array positioned on diametrically opposing sides of a rotating gantry. During a scan of an object, the object is placed in an examination region of the scanner and the gantry rotates about the object while radiation is emitted from a focal spot of the radiation source. Many conventional CT scanners use a collimator to shape the X-ray radiation beam emitted from the focal spot. Typically, the collimator directs the X-ray radiation beam in a direction generally perpendicular to an axis of rotation of the gantry (the z-axis) and toward the detector array. In some known scanners, the collimator forms a narrow beam, e.g. a fan beam that is shaped to impinge on one or several rows (slices) in the detector array. In other known scanners, the collimator forms a wider volume beam, e.g. a cone beam that is shaped to impinge a plurality of rows in the detector array.

U.S. Pat. No. 8,537,965 entitled “Cone-Beam CT,” assigned to Arineta Ltd., the content of which is incorporated herein by reference, describes an X-ray source system for a CT scanner that includes a plurality of X-ray sources spaced in a direction parallel to the axis of rotation (the z-axis), grid electrodes that selectively block radiation from the X-ray sources and a grid modulator configured to apply voltage to grid electrodes of each of the plurality of X-ray sources in turn.

U.S. Patent Application Publication No. 2011/0007878 entitled “Imaging System using Multisource Collimation and a Method Assembly and System for providing Multisource Collimation,” assigned to Arineta Ltd., the content of which is incorporated herein by reference, describes a collimator assembly for a multi-radiation-source medical imaging system (e.g. CT) and a medical imaging system utilizing the collimator. According to some embodiments of the present invention, there is provided a collimator assembly including at least two apertures, which apertures are adjustable substantially synchronously by one or more actuators.

Typically, a reconstruction process is used for reconstructing an image of a scanned object based on measured data values obtained during acquisition of multiple projection views. Typically, high geometrical consistency between the measured data obtained from different angles throughout the scan is required to achieve a reconstructed image to the desired level of quality. Consequently, any mechanical misalignments and/or displacement of the various tomography components during data acquisition can potentially cause inaccuracies and/or artifacts in the reconstructed images.

One known cause of misalignment is related to movement of the radiation source during scanning. During a scan, multiple forces, e.g. thermal, gravitational, and centrifugal may cause the x-ray source to shift. This shift results in inconsistency within the acquired data. For CT scanners with wide detector arrays and multiple radiation sources, significant errors are typically related to shifts along the z-axis. Known CT scanners typically track movement of the focal spot during scanning and provide compensation for the detected movement in the process of acquisition. Shifting of the X-ray source can also lead to misalignment with a collimator that shapes the X-ray beam. CT scanners that scan with narrow fan beams are known to be particularly sensitive to misalignment with the collimator in the Z direction. It is known to align position of collimator blades with the new focal spot position.

U.S. Pat. No. 5,550,886 entitled “X-ray focal spot movement compensation system,” describes an apparatus for compensating for drift of the focal spot of an X-ray radiation source used in a tomography system. The apparatus is used to maintain a primary collimated beam of radiation emanating from the focal spot aligned with target detectors of the tomography system. A second collimated beam of radiation is produced from the same focal spot and directed along a different axis from the first beam. An array of detectors tracks the movement of the second collimated beam and produces signals which are used to reposition the collimator used to collimate the primary beam so as to maintain the primary beam substantially aligned with the target detectors.

U.S. Pat. No. 5,610,967 entitled “X-ray grid assembly,” the content of which is incorporated herein by reference, describes a stepped scanning-beam x-ray source and a multi-detector array. The output of the multi-detector array is input to an image reconstruction engine which combines the outputs of the multiple detectors over selected steps of the x-ray beam to generate an x-ray image of the object. A collimating element, in the form of a perforated grid containing an array of apertures is interposed between the x-ray source and an object to be x-rayed. The function of the collimating element is to form thin pencil beams of x-rays, all directed from a focal spot on the anode target of the x-ray tube toward the multi-detector array. It is stated that the use of the collimating element provides for imaging with reduced X-ray dosage to patients and without loss of resolution.

U.S. Pat. No. 6,542,576 entitled “X-ray tube for CT applications” the content of which is incorporated herein by reference, describes an x-ray tube assembly including a vacuum envelope and an x-ray permeable exit window. An anode is positioned within the vacuum envelope such that a near side is adjacent to the exit window and a far side is opposite thereof. A set of radiation attenuating vanes, a filter and an externally located collimator provides for shaping the output x-ray beam. The x-ray tube housing optionally has an additional internal collimator aperture and window that along with a housing window allows radiation to impinge upon reference detector arrays that monitor the position of the x-ray focal spot in two dimensions. The detector arrays can be photodiodes, ion chambers or any x-ray sensitive devices usable to track and passively monitor the focal point of the electron beam or apex of the x-ray beam. The detectors also provide a reference radiation intensity value.

U.S. Pat. No. 7,284,905 entitled “X-ray radiator, x-ray device and computed tomography apparatus with focus position determining capability.” the content of which is incorporated herein by reference, describes an x-ray radiator has a radiator housing from which x-ray radiation originating from a focus is emitted. A pre-diaphragm is disposed in the beam path of the x-ray radiation and has a diaphragm opening in or on the radiator housing. The pre-diaphragm is provided with at least one additional slit through which x-ray radiation can strike on at least one element for determination of the position of the focus. It is described that the space requirement for the x-ray device is reduced relative to the conventional arrangement by providing the additional slit directly in or on the radiator housing of the x-ray radiator, rather than in a housing downstream from the x-ray radiator, as is conventional. A suitable separation relationship between the focus, the pre-diaphragm and the element for determination of the position of the focus is still required.

U.S. Pat. No. 7,778,384 entitled “Direct measuring and correction of scatter for CT,” the content of which is incorporated herein by reference, describes an examination apparatus including a radiation source adapted for emitting electromagnetic radiation to the object of interest, a detector unit adapted for detecting image data and scatter data from the object of interest, and a correction unit adapted for correcting the image data on the basis of the scatter data. The examination apparatus includes a one-dimensional anti-scatter-grid for filtering the electromagnetic radiation. The image data is detected at a first position of a focal spot of the electromagnetic radiation relative to the detector unit and the scatter data is detected at a second position of the focal spot relative to the detector unit. The anti-scatter-grid is adapted such that no direct radiation hits the detector unit when the electromagnetic radiation is focused to the second position of the focal spot.

### SUMMARY OF THE INVENTION

According to an aspect of some embodiments of the present invention there is provided a dedicated tracking system for tracking focal spot movement of a radiation source during scanning. In some exemplary embodiments, the dedicated tracking system is suitable for being positioned in close proximity to the radiation source. According to some embodiments of the present invention, the tracking system provides sub-pixel resolution, so that high accuracy tracking can be achieved with coarse and/or low accuracy detector array.

According to an aspect of some embodiments of the present invention there is provided a tracking system for tracking focal spot position of an x-ray source, the tracking system comprising: a detector array including a plurality of detecting elements sensitive to x-ray radiation; and a grid overlaid on a detecting surface of the detector array, wherein the grid is formed from an array of vanes and wherein a density of the grid is greater than a density of the detector array.

Optionally, the detector array is a one dimensional array and the array of vanes is a one dimensional array that is distributed across the array of detecting elements.

Optionally, the vanes in the array are parallel to each other.

Optionally, the vanes of the grid are orthogonal to the detecting surface of the detector array.

Optionally, the system includes a filter positioned over the detecting surface of the detector array and between the detector array and grid.

Optionally, a filter positioned over a surface of the grid that is distal to the detecting surface of the detector array.

Optionally, a ratio between a height of the vanes and a distance between contiguous vanes is defined to range from 8 to 15.

Optionally, the detector elements provide a 0.5-2 mm pixel resolution.

Optionally, the system includes a processing unit for tracking focal spot position or focal spot movement based on output sampled from the detector array.

According to an aspect of some embodiments of the present invention there is provided an x-ray device comprising: an x-ray source emitting x-ray radiation from a focal spot; a collimator collimating radiation emitted from the x-ray source; and a tracking system as described herein above receiving a portion of the radiation of the x-ray source does not penetrate through the collimator.

Optionally, the tracking system is positioned 5-15 cm from the focal spot.

Optionally, the tracking system receives the first portion of radiation directly from the focal spot without magnification of the focal spot.

Optionally, the x-ray source emits x-ray radiation from two focal spots and wherein the tracking system receives the first portion of radiation from one of the two focal spots.

Optionally, the x-ray device is retrofitted with the tracking system.

Optionally, the collimator is adjustable and is adapted to follow the focal spot position as detected by the tracking system.

Optionally, the device includes a second focal spot from which x-ray radiation is emitted and a second collimator collimating radiation from the second focal spot.

Optionally, the device includes a second tracking system receiving a portion of the radiation that does not penetrate through the second collimator.

Optionally, the second focal spot is provided with a second x-ray source.

According to an aspect of some embodiments of the present invention there is provided CT scanner comprising: a gantry, wherein the gantry houses: a rotating frame that rotates about a Z axis; an x-ray device as described herein above, wherein the x-ray device is mounted on the rotating frame; and a scanning detector array, wherein the scanning detector array is mounted on the rotating frame and opposite the x-ray device; and a controller that controls operation of the CT scanner.

Optionally, the array of vanes of the grid is aligned to parallel with the Z axis.

Optionally, the scanner includes a second x-ray device according, wherein the second x-ray device is mounted on the rotating frame.

Optionally, the controller is operative to receive input from the tracking system of the x-ray device and to adjust reconstruction of images generated from output obtained from the scanning detector array based on the input received.

According to an aspect of some embodiments of the present invention there is provided method for tracking position of a focal spot of an x-ray source, the method comprising: detecting radiation emitted from a focal spot with an array of x-ray sensitive detectors, wherein the radiation detected is radiation that passed through an array of vanes and wherein a density of the array of vanes is greater than a density of the array of x-ray sensitive detectors; modeling output from the detector array; identifying a peak in the model of the output; and associating position of the focal spot with the peak of the model.

Optionally, the method includes positioning the array of x-ray sensitive detectors at distance of 5 to 15 cm from the focal spot.

Optionally, the method includes detecting radiation with the array of x-ray sensitive detectors absent magnification of the focal spot.

Optionally, the method includes adjusting a collimator associated with the x-ray source responsive to a current position of the focal spot.

Optionally, the method includes adjusting image reconstruction of images captured responsive to a current position of the focal spot.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A and 1B are simplified schematic drawings of an exemplary CT scanner including a tracking system shown in a front and side view respectively in accordance with some embodiments of the present invention;

FIG. 2 is a simplified schematic drawing of tracking system in accordance with some embodiments of the present invention;

FIG. 3 is a simplified graph showing intensity sensed by a detector array of a tracking system without a grid and with a grid in accordance with some embodiments of the present invention;

FIG. 4 is a simplified graph showing intensities curves obtained at two different focal spot positions in accordance with some embodiments of the present invention;

FIG. 5 is a simplified flow chart of an exemplary method for focal spot tracking in accordance with some embodiments of the present invention;

FIG. 6 is a simplified schematic drawing of an exemplary grid for a tracking system in accordance with some embodiments of the present invention;

FIG. 7 is a simplified schematic drawing of a grid and detector array in accordance with some embodiments of the present invention;

FIG. 8A is a simplified schematic drawing showing an exemplary geometry for a detector array that includes a sub-pixel shift between rows of the detector array in accordance with some embodiments of the present invention.

FIGS. 8B and 8C are simplified schematic drawings showing two exemplary geometries for a detector array that provides two dimensional tracking in accordance with some embodiments of the present invention; and

FIGS. 9A and 9B are simplified schematic drawings of two exemplary multi-focal spot X-ray devices that are integrated with a tracking system in accordance with some embodiments of the present invention.

### DESCRIPTION OF SPECIFIC EMBODIMENTS OF THE INVENTION

The present invention, in some embodiments thereof, relates to focal spot position tracking of a radiation source and, more particularly, but not exclusively, to focal spot position tracking for radiation scanners, such as CT scanners and the like.

U.S. Pat. No. 7,284,905 referenced hereinabove describes focal spot tracking by projection of the focal spot image through a slit or a pin hole onto an X-ray sensitive sensor and detection of motion of the projected focal spot image on the sensor. The slit cannot typically be placed very close to the focal spot because of constrains of the radiation source structure which further distances he X-ray sensitive sensor from the focal spot. On the other hand, to get high sensitivity to focal spot motion it is desired to have high magnification, achievable by placing the sensor as far as practical from the slit. Thus, prior art solutions are feasible only if sufficient distance can be provided between the elements of the tracking system.

According to an aspect of some embodiments of the present invention, there is provided a tracking system that provides high accuracy focal spot tracking without requiring a slit or a pin hole to project focal spot image with magnification on the sensor. In some exemplary embodiments, the tracking system provides a desired tracking resolution without requiring substantial and/or any magnification. In some exemplary embodiments, the tracking system is suitable for compact CT scanners whose limited space within the gantry does not accommodate for substantial magnification of the focal spot by distancing the reference detector array.

According to some embodiments of the present invention, the improved resolution is obtained by adding and/or placing a grid between the X-ray source and the detector array for focal spot tracking. Optionally, the grid includes a dense set of parallel strips, vanes, septa and/or leaves. Typically, the grid has the effect of reducing intensity of the flux on the detector plane as a function of distance from an orthogonal projection of the focal spot. Due to this effect, peak intensity on the detector plane is typically received by a detector element(s) aligned with the orthogonal projection of the focal spot center while detector elements distanced from the orthogonal projection record significantly lower intensity. The present inventors have found that the imposed gradient can be harnessed for increasing the resolution for locating position of the focal spot and thereby enhancing sensitivity of the tracking system to changes in the focal spot location. In some exemplary embodiments, the tracking system includes and/or is associated with a processor for tracking position and/or movement of a focal spot based on output detected from the detector array of the tracking system. Optionally, model-based maximum likelihood position estimation is used to track focal spot location.

According to some embodiments of the present invention, the grid is defined to be denser than a pitch, e.g. pixel pitch of the detector array and the imposed gradient provides for tracking of the focal spot location with sub-detector-pixel resolution. The present inventors have found that the tracking system as described herein can provide high accuracy measurements with minimal space requirements. In addition, the present inventors have found that the tracking system as described herein is advantageous in that high accuracy measurements can be obtained with lower resolution detector arrays. The reduced resolution detector array provides for both reducing cost and reducing the amount of computations required when processing output from the array.

According to some embodiments of the present invention, a ratio, e.g. an optimized ratio between septa height and aperture width of the grid is selected to obtain a desired gradient. In some embodiments, the tracking system additionally includes an attenuating hardware filter positioned on top and/or bottom of the grid. Optionally, the filter is used to reduce the relatively high X-ray flux intensity on the detector element due to the proximity of the detector element to the source. Optionally, the filter provides structural rigidity for the tracking system. The grid can be a one-dimensional grid or a two-dimensional grid.

According to some embodiments of the present invention, focal spot position and/or shift in the focal spot position is fed into the image reconstruction algorithms for more accurate reconstruction, e.g. is used in the back projection process for reconstructing images. In some exemplary embodiments, focal spot position is used to adjust position of the beam collimator to that follows the actual position of the focal spot. Optionally, a collimator assembly receives input from a tracking system and/or is adjusted based on output detected from a tracking system. Typically, adjusting position of the beam collimator is performed on CT scanners that scan with relatively narrow beams.

Before explaining at least one embodiment of the invention in detail, it is to be understood that the invention is not necessarily limited in its application to the details of construction and the arrangement of the components and/or methods set forth in the following description and/or illustrated in the drawings. The invention is capable of other embodiments or of being practiced or carried out in various ways.

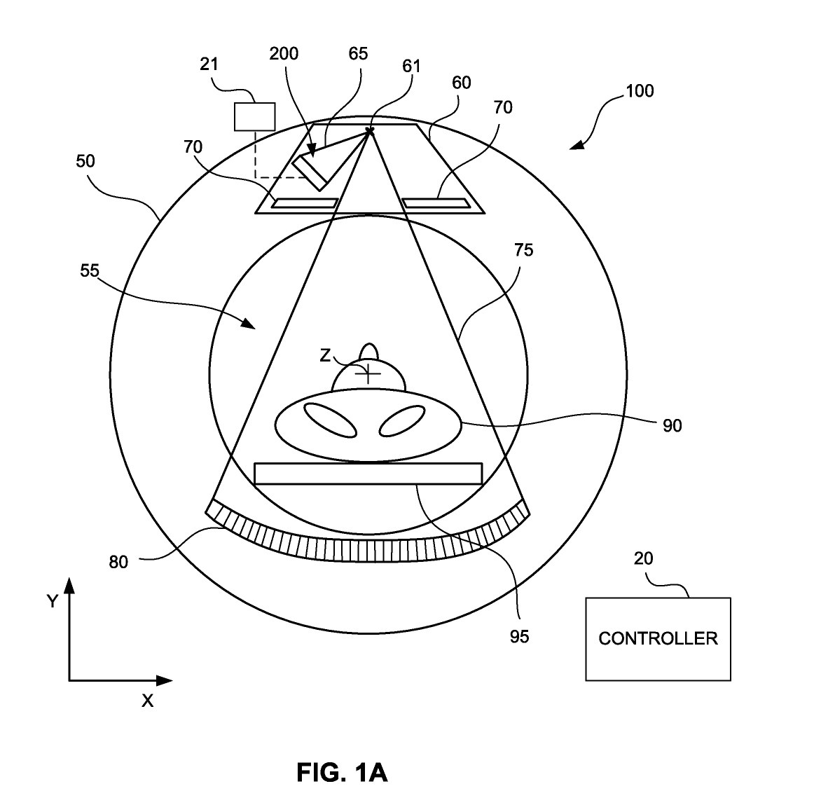

Referring now to the drawings, FIGS. 1A and 1B show simplified schematic drawings of an exemplary CT scanner including a tracking system shown in a front and side view respectively in accordance with some embodiments of the present invention. A CT scanner 100 includes a gantry 50 and a support platform 95 extending into a bore 55 of gantry 50, on which a patient 90 or object is supported during an examination procedure. Optionally, support platform 95 is movable and is controllably displaced in a direction parallel to a Z axis direction, during examination.

Typically, gantry 50 houses an x-ray device 60 that emits one or more x-ray beams from one or more focal spots, e.g. focal spot 61, and an x-ray detector 80 that captures radiation emitted from x-ray device 60 after being attenuated by patient 90.

Typically, x-ray detector 80 includes a plurality of detecting elements arranged in a one or two dimensional array. Typically, during examination both x-ray device 60 and x-ray detector 80 are rotated within gantry 50 about axis Z, typically, centered with bore 55. Typically, CT scanner 100 is associated with a controller and/or processing unit 20 and/or computing device that controls operation of the CT scanner.

According to some embodiments of the present invention controller and/or processing unit 20 includes and/or is associated with an image reconstruction engine for constructing images from data captured.

Typically, x-ray device 60 includes a collimator 70 for shaping beam 75 into a desired shape, e.g. fan or cone shape. Beam 75 is typically formed from a portion of the x-rays emitted from focal spot 61. According to some embodiments of the present invention, x-ray device 60 additionally includes and/or is retrofitted with a tracking system 200 for tracking one or more focal spots 61 during an examination procedure. According to some embodiments of the present invention, output from tracking system 200 is used by controller and/or processing unit 20 for computing image reconstruction and/or for adjusting collimator 70 so that it follows focal spot 61.

According to some embodiments of the present invention, tracking system 200 is configured for being positioned in close proximity to focal spot 61, e.g. within 5-10 cm or 5-20 cm. Optionally, tracking system 200 is positioned between focal spot 61 and collimator 70 and/or closer to focal spot 61 as compared to collimator 70. Optionally, tracking system is fixed onto a rigid construction associated with collimator 70. Alternatively, tracking system 200 is further distanced from focal spot 61.

According to some embodiments of the present invention, tracking system 200 detects a beam 65, e.g. a portion of the x-rays emitted from focal spot 61 that extends angularly outside the range of beam 75 shaped by collimator 70, and tracks focal spot 61 based on input from beam 65. Optionally, beam 65 is not shaped by a collimator and/or dedicated aperture.

Typically, beam 65 from focal spot 61 is separate from or other than beam 75. In some exemplary embodiments, beam 75 shaped by collimator 70 spans about 60° while a beam emitted from focal spot 61 spans an angle of about 80°. According to some embodiments of the present invention, tracking system 200 is placed about 8-10 cm from the focal spot in for example a peripheral 10° the beam emitted by focal spot 61 and/or between 30° to 40° from centerline of the beam emitted from focal spot 61. According to some embodiments of the present invention, a sensing surface of tracking system 200 is positioned so that line normal to the sensing surface extends to focal point 61. In some exemplary embodiments, an existing CT scanner is retrofitted with tracking system 200. Typically, controller 20 and/or an associated computing device controls operation and/or processes output of tracking system 200. Optionally, tracking system 200 is associated with a dedicated processor and/or controller 21 in communication with controller 20. Typically, movement of focal spot 61, e.g. to position 61′ is detected by controller 21 of tracking system 200 (FIG. 1B) when present. Alternatively, focal spot position and/or movement are tracked by controller and/or processing unit 20.

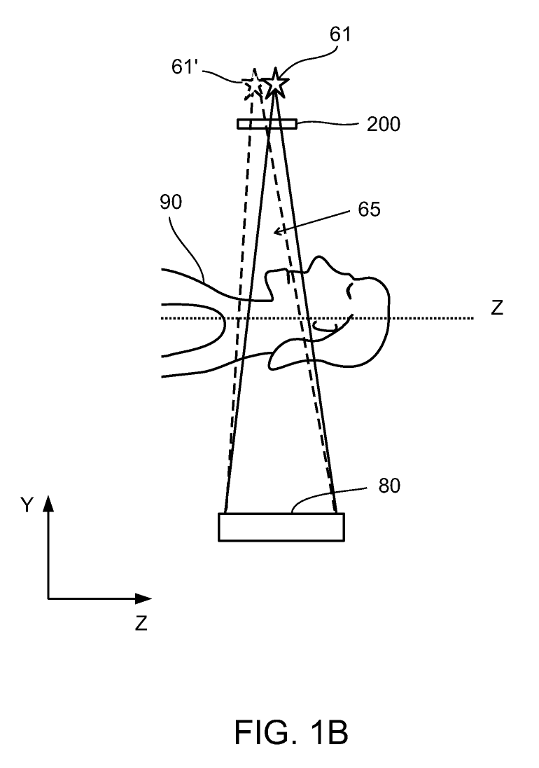

Reference is now made to FIG. 2 showing a simplified schematic drawing of a tracking system 200 in accordance with some embodiments of the present invention. According to some embodiments of the present invention, resolution for tracking focal spot is improved by using a grid 260, e.g. a one dimensional grid including a plurality of partitions and/or leaves and positioning grid 260 between focal spot 61 and focal spot tracking detector array 250. Typically, the leaves of grid 260 are made of high attenuation material such as lead or tungsten or other material whereas the space between leaves is filled with low attenuation material such as aluminum, plastic, air or other material. Typically, grid 260 is overlaid on a detecting plane of detector array 250. According to some embodiments of the present invention, grid 260 includes an array of parallel partitions that are aligned with an orthogonal projection of focal spot 61 on a detecting plane of detector array 250. Typically, direct penetration of beam 65 through grid 260 is achieved mostly in portions of grid 260 where the angle of rays in beam 65 are more aligned with the partitions of grid 260, e.g. partitions 261, while less penetration occurs in portions of grid 260 where the rays are more angled with respect to the partitions, e.g. partitions 262. Typically, for grid 260 that includes a set of orthogonally positioned partitions, the best alignment between beam 65 and grid 260 occurs in an area around the orthogonal projection of focal spot 61 on grid 260, while reduced penetration occurs in the periphery. This is typically due to the fan and/or cone shape of beam 65.

Typically, detector array 250 includes an array of detecting elements 251. According to some embodiments of the present invention, the array of parallel partitions of grid 260 is denser than the array of detecting elements 251 so that each detecting element 251 receives radiation through more than one set of partitions of grid 260. The present inventor has found that when using grid 260, intensity of radiation detected on detecting elements 251 substantially aligned with the orthogonal projection, e.g. detecting elements 4 and 5 is significantly higher than the intensity detected on neighboring detecting elements 251, e.g. detecting elements 1, 2, 3 and detecting elements 6, 7, 8. Typically, as the alignment between rays of beam 65 and partitions of grid 260 decrease, the intensity detected by the under laying detecting elements 251 also decrease. Typically, the intensity decreases significantly with distance from the orthogonal projection and as the angle of the ray deviates from angle of the partitioning element. The present inventors have found that by shaping the output in this manner, additional information is added to the output and that additional information can be used to identify focal spot position or shifts. It is noted that although most of the embodiments of the present invention, are defined with respect to a grid that includes a parallel partitions that are orthogonal with respect to detector array 250, the invention is not limited in this respect and other angles for the partitions can be used.

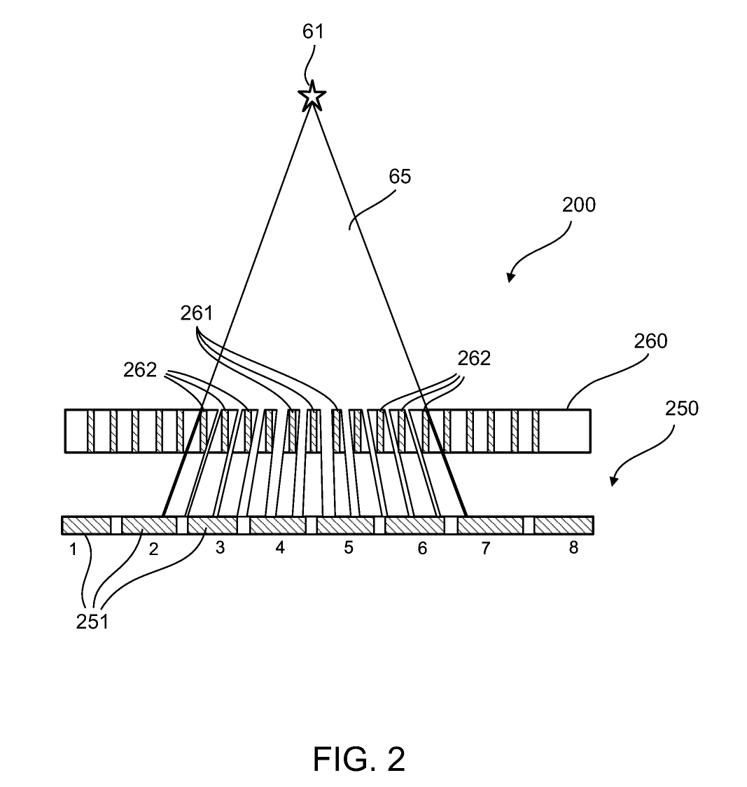

Reference is now made to FIG. 3 showing graph of intensity sensed by a detector array of a tracking system without a grid and with a grid in accordance with some embodiments of the present invention. In the exemplary graph shown, intensity across sixteen detecting elements is shown. Outputs 310 represent output obtained when grid 260 is not used. Typically, when grid 260 is not used, a relatively constant intensity is detected on a plurality of detecting elements 251 near the orthogonal projection of focal spot 61. According to some embodiments of the present invention, output 320 is obtained when using grid 260. Typically, grid 260 imposes a slope on both sides of the focal spot position so that the highest intensity is detected at or close to an orthogonal projection of the focal spot and lower intensity is detected away from the orthogonal projection of the focal spot. Typically, intensity decrease as a function of distance from the orthogonal projection of the focal spot position. The present inventors have found that when shaping the output in this manner, to obtain output 320, the focal spot position can be more easily identified and tracked. Typically, output sampled from a plurality of detecting elements is used to estimate the focal spot position. According to some embodiments of the present invention, a curve 321 is fitted to the output detected on each detecting element and the focal spot position is associated with location of a peak 325 of curve 321. It is noted that the output 310 and 320 represent a particular example of with detecting elements 251 positioned 13 cm from a focal spot. If is further noted that curve 321 represents output when a grid of more than 50 strips per cm was used over the array sixteen detector elements. It is noted, the number of detecting elements included in the array, the distance from the focal spot and the density of the collimator grid is only exemplary and can be altered depending on the application and resolution required.

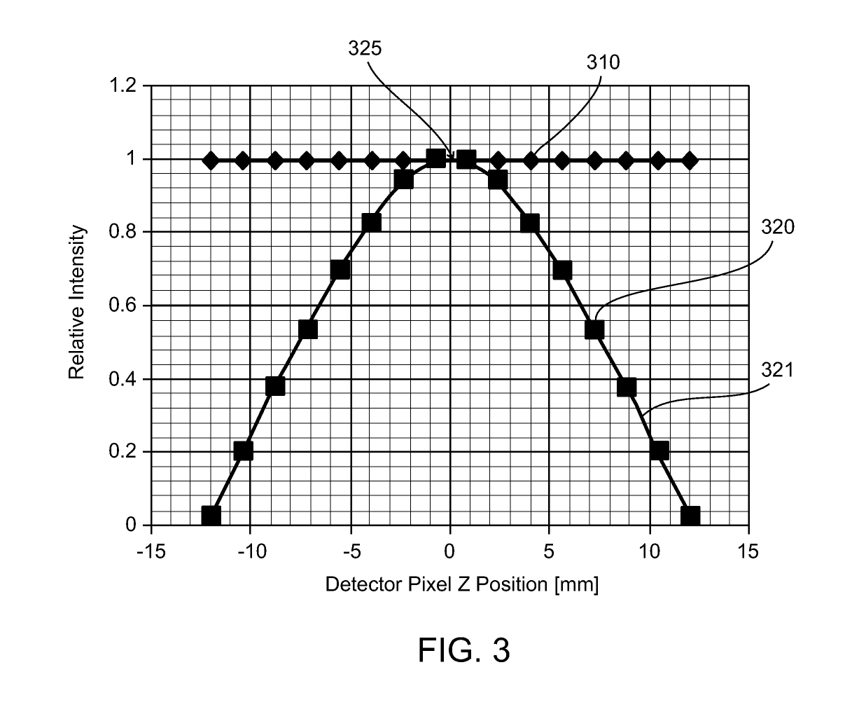

Reference is now made to FIG. 4 showing a simplified graph with intensities curves obtained at two different focal spot positions in accordance with some embodiments of the present invention. Outputs 320 and 420 represent output at two different focal spot positions. According to some embodiments of the present invention, curves 321 and 421 are fitted to outputs 320 and 420 respectively. In the particular example shown, there is a 0.2 mm shift in the focal spot position while the pixel resolution provided by the detecting elements is about 1 mm. According to some embodiments of the present invention, sub-pixel shifts in focal spot positions can be detected by comparing curves 321 and 421, e.g. comparing peaks of curves 321 and 421. Typically, a shift in a curve fitted to the output and/or a position of a peak of the curve respectively corresponds to the shift and/or position of the focal spot.

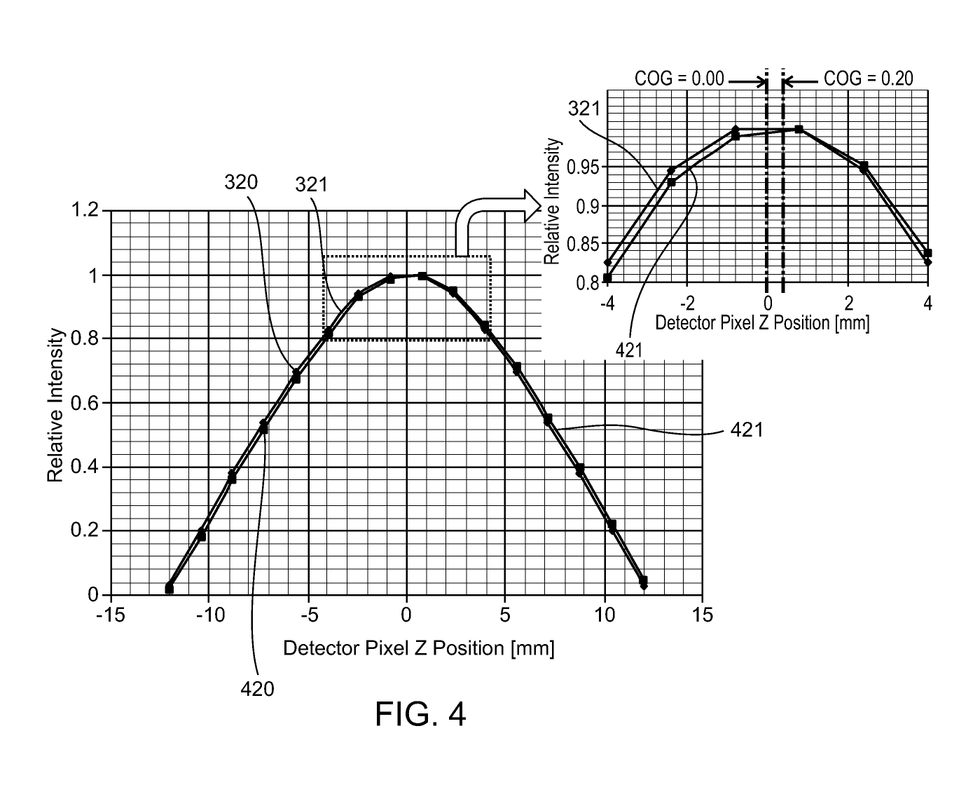

Reference is now made to FIG. 5 showing a simplified flow chart of an exemplary method for focal spot tracking in accordance with some embodiments of the present invention. According to some embodiments of the present invention, during an examination procedure with CT scanner 100, output from a detector array 250 of a tracking system 200 is sampled (block 510). According to some embodiments of the present invention, the output sampled is modeled, e.g. a curve is fitted to the output sampled (block 520). In some exemplary embodiments, linear interpolation, center of gravity calculation, maximum likelihood estimation (MLE), or model based estimation may be used to detect a peak in the output and/or a shift in the peak. In some exemplary embodiments, a polynomial equation is used to model an intensity curve using discrete intensities detected by the detector array. According to some exemplary embodiments, based on the model used, a peak in the output obtained across the array is identified (block 530) and the position of the peak output is reported to and/or used by controller 20 of CT scanner 100 for controlling operation of CT scanner 100 and/or processing the output from CT scanner 100. Other embodiments may rely on other parameters, such as signal slopes, or alternatively, overall signal shape. According to some embodiments of the present invention, focal spot position and/or shift in the focal spot position is fed into the image reconstruction algorithms for more accurate reconstruction, e.g. is used in the back projection process for reconstructing images. In some exemplary embodiments, focal spot position is used to adjust position of the beam collimator to follow the actual position of the focal spot. Typically, adjusting position of the beam collimator is performed on CT scanners that scan with relatively narrow beams.

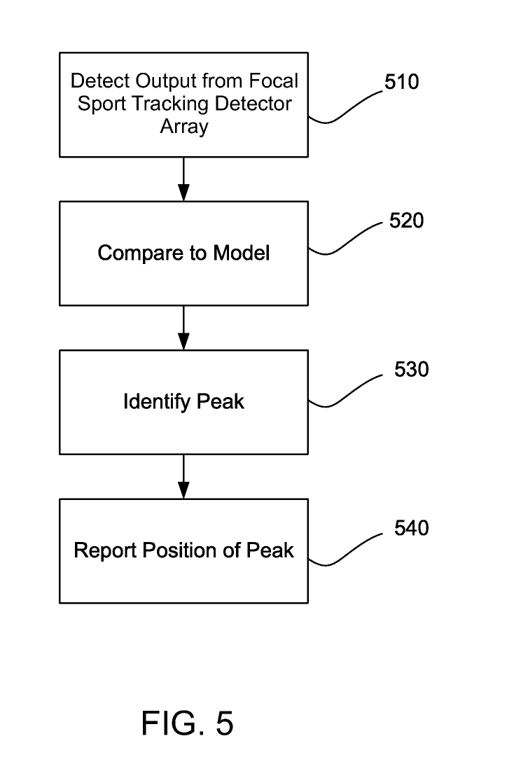

Reference is now made to FIG. 6 a simplified schematic drawing of an exemplary grid for a tracking system in accordance with some embodiments of the present invention. In some exemplary embodiments, grid 260 is a one dimension grid that is formed with a plurality of lead strips. Typically, the height of the lead strips, ‘H,’ and/or density of the grid, e.g. number of strips per mm define the shape of the output, e.g. the slope of the bell shaped and/or parabolic shaped curve. In some exemplary embodiments, the array of lead strips is aligned along a Z axis direction so that focal spot shift in the Z axis direction can be detected.

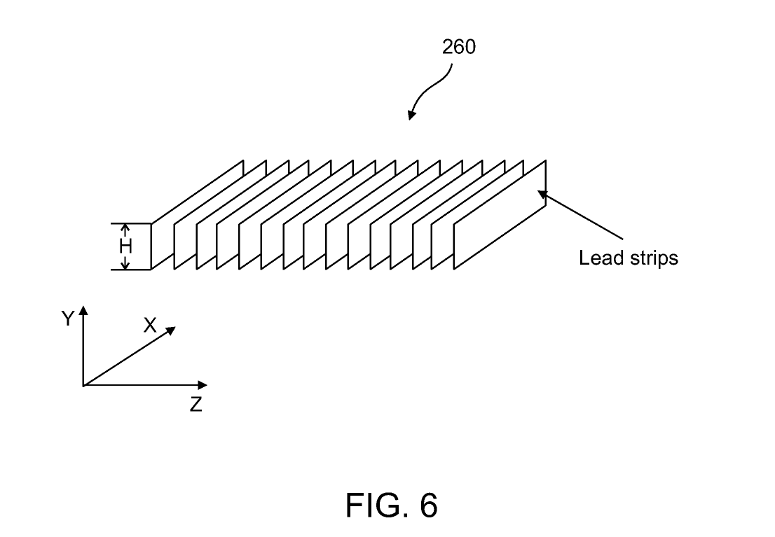

Reference is now made to FIG. 7 showing a simplified schematic drawing of a grid and detector array in accordance with some embodiments of the present invention. According to some embodiments of the present invention, the grid provides for tracking focal spot at close distances with a relatively low resolution and low cost detector array 250. Optionally, relatively large detecting elements 251 providing for example a 1 mm pixel resolution are used to detect sub-pixel shifts in focal spot. Optionally, low cost detector arrays including significant gaps 281 between detector elements 251 are used and sub-pixel resolution is still obtained. In some exemplary embodiments, the partitions and/or leaves are defined to have a height, ‘H’ of between 1.5-2.5 mm, e.g. 1.68 or 2.3 mm. Optionally, the thickness of a leaf is around 0.05 mm and distance between leaves is 0.1-0.3, e.g. 0.15-0.2 or 0.17 mm. In some exemplary embodiments, for a tracking system positioned about 10 cm from a focal spot, a grid density of about 8 leaves/mm is used with a ratio between height and distance between leaves, defined to fall within the following range:

$$8 \leq \frac{H}{D} \leq 15 \qquad Equation ( 1 )$$

Optionally, when using the ratio as defined in Equation (1), dome or parabolic shaped curve is obtained from the x-ray tube output. Optionally, other configurations can be defined depending on the required resolution, the resolution provided by the detector array 250, the H/D ratio and the distance from the focal spot. Optionally, a hardware filter 280 is positioned over grid 260, e.g. over the surface of the grid facing the focal spot, between detector array 250 and grid 260 or both. Optionally, hardware filter 280 is used to provide rigidity to tracking system 200. Optionally, hardware filter 280 is used to reduce the intensity of the radiation reaching detector array 250. Typically, when tracking a focal spot at close proximity to the focal spot, the radiation is intense and may need to be reduced to protect detector array 250. Alternatively, grid 260 is used with a high resolution detector array, e.g. to improve available resolution. Optionally the grid spacing and detector resolution are adjusted according to the width of the focal spot, e.g. for a narrower focal spot, higher grid density and higher resolution detector are used.

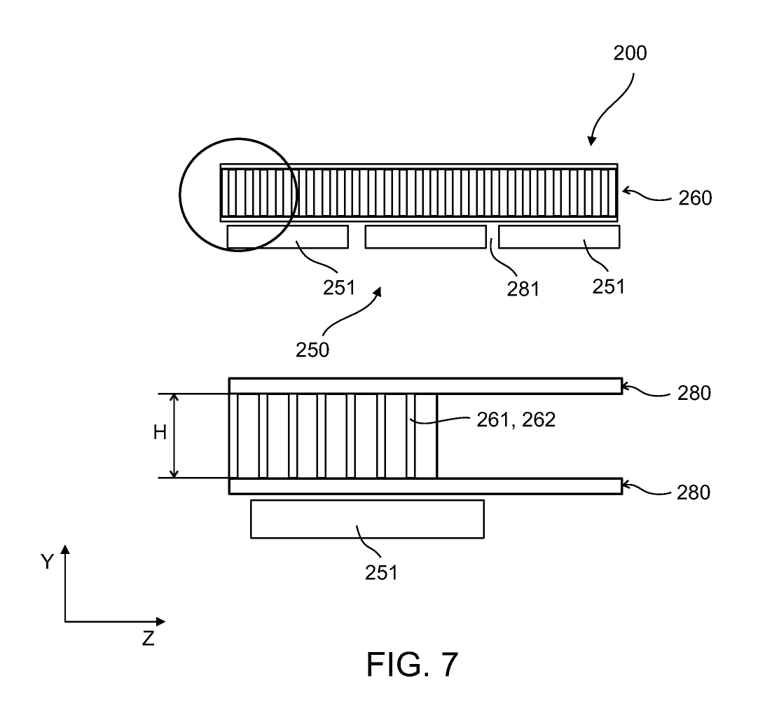

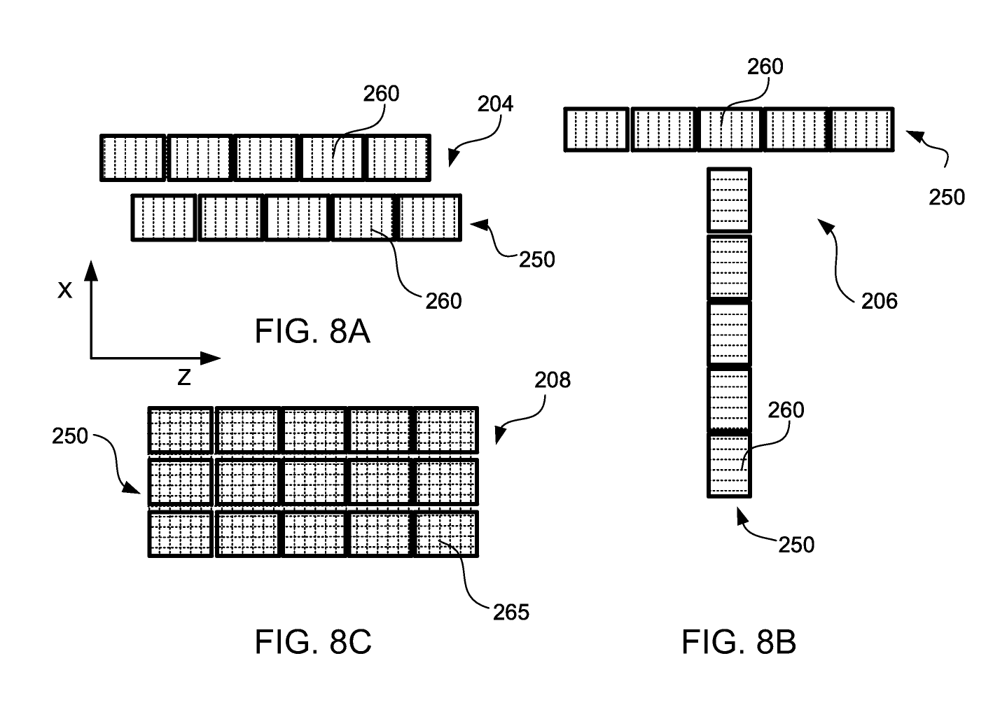

Reference is now made to FIG. 8A showing a simplified schematic drawing of an exemplary geometry for a detector array that includes a sub-pixel shift between rows of the detector array in accordance with some embodiments of the present invention. According to some embodiments of the present invention, a tracking system includes a plurality of one dimensional detector arrays and/or a two dimensional grid of detectors. Optionally, tracking system 204 includes a pair (or a plurality) of one dimensional arrays 250, both (or all) aligned in a Z axis direction with a half a pixel shift (or sub-pixel shift) between them. Optionally, such a configuration provides for increasing the resolution for detecting shift in the Z axis direction. Typically, each detector array is associated with a dedicated grid 260. Typically, the leaves of grid are distributed along the Z axis direction and/or the direction along which the detecting elements of detector array 250 are distributed. It is noted that detecting elements are represented as rectangles while, grid, e.g. leaves of grid 260 are represented here as a set of dotted lines. The dotted lines are used for clarity purposes.

Reference is now made to FIGS. 8B and 8C showing simplified schematic drawings of two exemplary geometries for a detector array that provides two dimensional tracking in accordance with some embodiments of the present invention.

Referring now to FIG. 8B, optionally, tracking system 206 includes a pair of one dimensional detector arrays 250 positioned orthogonally for one another. In some exemplary embodiments, each detector array 250 of the pair is associated with a dedicated grid 260 distributed along a length of detector array 250, e.g. with leaves or partitions of grid 260 distributed along a length of detector array 50. Referring now to FIG. 8C, optionally, a plurality of one dimensional detector arrays 250 form a tracking system 208 include a two dimensional grid detector array. Optionally, the two dimensional detector array detects shifts in the focal spot in both the X axis and Z axis direction. Optionally, the two dimensional detector array is also used to track spot size. In some exemplary embodiments, a two dimensional grid 265 is used for tracking system 208. Optionally, the two dimensional grid is denser than the detector array in both the first and second dimension, e.g. X and Z axis direction so that sub-pixel resolution can be obtained in two dimensions. In some exemplary embodiments, the two dimensional grid is mesh formed from lead strips.

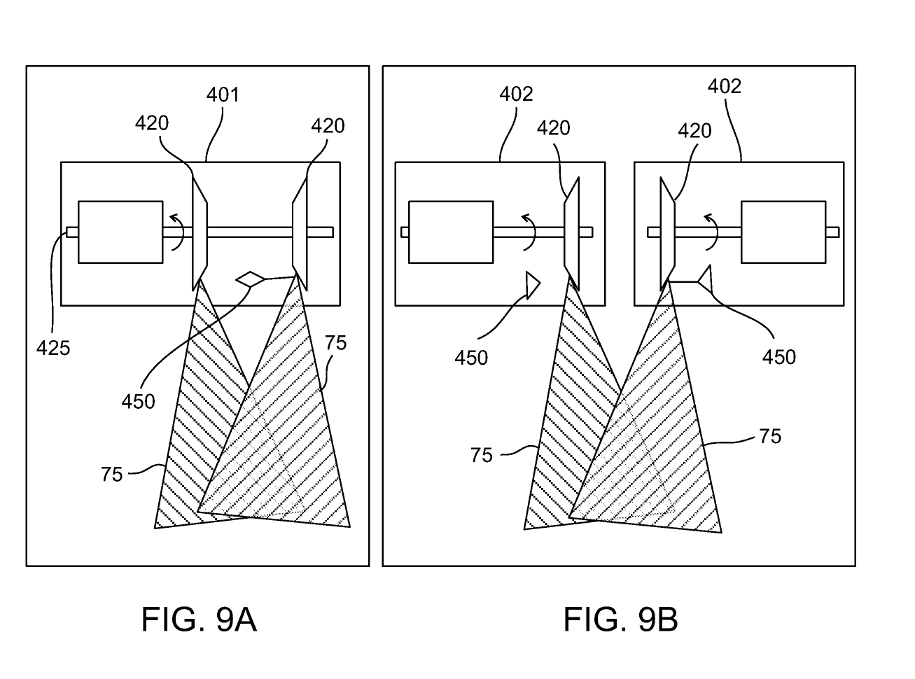

Reference is now made to FIGS. 9A and 9B showing simplified schematic drawings of two exemplary multi-focal spot X-ray devices that are integrated with a tracking system in accordance with some embodiments of the present invention. In some exemplary embodiments, a CT scanner includes an x-ray source 401 that provides more than one beam 75 or a plurality of x-ray sources 402, each of which provide a single beam 75. Optionally, a CT scanner includes a plurality of x-ray sources 402. In some exemplary embodiments, when a same x-ray source 401 is used to provide more than one beam 75, a common cathode 450 is used and each beam is emitted from a dedicated anode 420. Typically, the anodes 420 are connected to a same shaft 425 so that when a shift in focal spot occurs, a same shift may be recorded in each of the focal spots. In some exemplary embodiments, when anodes 420 are connected to same shaft 425, shift in focal spot may be detected from only one of the focal spots and the other focal spot is assumed to shift in the same manner.

Alternatively, in some cases thermal expansion of the shaft may lead to variations in shifts of the focal spots. Other embodiments that use multiple x-ray sources as shown in FIG. 9B typically require dedicated tracking for each X-ray source. Typically, when multiple x-ray sources are used, a dedicated tracking system 200 is required for each x-ray source 402.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

What is claimed is:

1. A tracking system for tracking focal spot position of an x-ray source, the tracking system comprising:
a detector array including a plurality of detecting elements sensitive to x-ray radiation; and
a grid overlaid on a detecting surface of the detector array, wherein the grid is formed from an array of vanes and wherein a density of the grid is greater than a density of the detector array.

2. The tracking system according to claim 1, wherein both the detector array is a one dimensional array and the array of vanes is a one dimensional array that is distributed across the array of detecting elements.

3. The tracking system according to claim 1, wherein the vanes in the array are parallel to each other.

4. The tracking system according to claim 1, wherein the vanes of the grid are orthogonal to the detecting surface of the detector array.

5. The tracking system according to claim 1, comprising a filter positioned over the detecting surface of the detector array and between the detector array and grid.

6. The tracking system according to claim 1, comprising a filter positioned over a surface of the grid that is distal to the detecting surface of the detector array.

7. The tracking system according to claim 1, wherein a ratio between a height of the vanes and a distance between contiguous vanes is defined to range between 8-15.

8. The tracking system according to claim 1, wherein the detector elements provide a 0.5-2 mm pixel resolution.

9. The tracking system according to claim 1, comprising a processing unit for tracking focal spot position or focal spot movement based on output sampled from the detector array.

10. An x-ray device comprising:
an x-ray source emitting x-ray radiation from a focal spot;
a collimator collimating radiation emitted from the x-ray source; and
a tracking system according to claim 1 receiving a portion of the radiation of the x-ray source does not penetrate through the collimator.

11. The x-ray device according to claim 10, wherein the tracking system is positioned 5-15 cm from the focal spot.

12. The x-ray device according to claim 10, wherein the tracking system receives the first portion of radiation directly from the focal spot without magnification of the focal spot.

13. The x-ray device according to claim 10, wherein the x-ray source emits x-ray radiation from two focal spots and wherein the tracking system receives the first portion of radiation from one of the two focal spots.

14. The x-ray device according to claim 10, wherein the x-ray device is retrofitted with the tracking system.

15. The x-ray device according to claim 10, wherein the collimator is adjustable and is adapted to follow the focal spot position as detected by the tracking system.

16. The x-ray device according to claim 10, comprising:
a second focal spot from which x-ray radiation is emitted; and
a second collimator collimating radiation from the second focal spot.

17. The x-ray device according to claim 16, comprising a second tracking system receiving a portion of the radiation that does not penetrate through the second collimator, wherein the second tracking system comprises:
a detector array including a plurality of detecting elements sensitive to x-ray radiation; and
a grid overlaid on a detecting surface of the detector array, wherein the grid is formed from an array of vanes and wherein a density of the grid is greater than a density of the detector array.

18. The x-ray system according to claim 16, wherein the second focal spot is provided with a second x-ray source.

19. A CT scanner comprising:
a gantry, wherein the gantry houses:
a rotating frame that rotates about a Z axis;
an x-ray device comprising:
an x-ray source emitting x-ray radiation from a focal spot;
a collimator collimating radiation emitted from the x-ray source;
and
a tracking system according to claim 1 receiving a portion of the radiation of the x-ray source does not penetrate through the collimator,
wherein the x-ray device is mounted on the rotating frame; and
a scanning detector array, wherein the scanning detector array is mounted on the rotating frame and opposite the x-ray device; and
a controller that controls operation of the CT scanner.

20. The scanner according to claim 19, wherein the array of vanes of the grid is aligned to parallel with the Z axis.

21. The scanner according to claim 19, comprising a second x-ray device, the second x-ray device comprising:
a second x-ray source emitting x-ray radiation from a focal spot;
a collimator collimating radiation emitted from the second x-ray source; and
a second tracking system comprising:
a second detector array including a plurality of detecting elements sensitive to x-ray radiation; and
a grid overlaid on a detecting surface of the second detector array,
wherein the grid is formed from an array of vanes and wherein a density of the grid is greater than a density of the detector array,
wherein the second tracking system receives a portion of the radiation of the x-ray source does not penetrate through the collimator and wherein the second x-ray device is mounted on the rotating frame.

22. The scanner according to claim 19, wherein the controller is operative to receive input from the tracking system of the x-ray device and to adjust reconstruction of images generated from output obtained from the scanning detector array based on the input received.

23. A method for tracking position of a focal spot of an x-ray source, the method comprising:
detecting radiation emitted from a focal spot with an array of x-ray sensitive detectors, wherein the radiation detected is radiation that passed through an array of vanes and wherein a density of the array of vanes is greater than a density of the array of x-ray sensitive detectors;
modeling output from the detector array;
identifying a peak in the model of the output; and
associating position of the focal spot with the peak of the model.

24. The method according to claim 23, comprising:
positioning the array of x-ray sensitive detectors at distance of between 5-15 cm from the focal spot.

25. The method according to claim 23, comprising detecting radiation with the array of x-ray sensitive detectors absent magnification of the focal spot.

26. The method according to claim 23, comprising adjusting a collimator associated with the x-ray source responsive to a current position of the focal spot.

27. The method according to claim 23, comprising adjusting image reconstruction of images captured responsive to a current position of the focal spot.
